(19) United States
(12) Patent Application Publication
(10) Pub. No.: US 2025/0186661 A1
(43) Pub. Date: Jun. 12, 2025

(54) LACTATION FLOW METER GAUGE, LACTATION-LATCH SENSOR DEVICE AND REMOVABLE FILTER

(71) Applicant: Jenny Lynn Walding, Palmyra, VA (US)

(72) Inventor: Jenny Lynn Walding, Palmyra, VA (US)

(21) Appl. No.: 18/977,632

(22) Filed: Dec. 11, 2024

Related U.S. Application Data
(60) Provisional application No. 63/609,111, filed on Dec. 12, 2023.

(51) Int. Cl. A61M 1/06

(52) U.S. Cl. CPC A61M 1/06; A61M 2205/3334; A61M 2205/7545

(57) ABSTRACT

A lactation flow meter device comprising a nipple shield having a lactation flow meter gauge, a lactation-latch sensor or plurality of sensors, and a removable filter or binder. The device includes a processor, battery or power source, and memory storage for receiving and processing data from the sensor or sensors. A removable filter assembly configured for use with a vessel. The filter assembly includes a straw portion, a filtering compression mechanism, a filter, or any combination thereof. The device is configured to communicate with an external personal device for tracking and processing data collected from the sensors. The nipple shield being customizable to a shape and size of an individual user. A method of manufacturing the device.

### BACKGROUND

### Technical Field

The present disclosure is directed to a lactation flow meter gauge, lactation-latch sensor device, and a removable filter.

### Description of the Related Art

Mother's milk (breast milk) is considered the best food for a newborn, being one of the essential elements for physical growth, immunological function, and psychological development of children, especially during the first year of life.

One of the most highly effective preventive measures a mother and parent can take to protect the health of her infant and herself is to breastfeed. However, in the U.S., while 83 percent of mothers start out breastfeeding, only 25 percent of babies are exclusively breastfed through six months. Additionally, these breastfeeding rates are significantly lower for infant minorities, specifically BIPOC (black, Indigenous, and people of color).

While breastfeeding is natural, it doesn't mean that it comes naturally. Breastfeeding is a skill both the mother and the newborn learn during this early life-critical time. Beyond misperceptions of feasibility, there is also the question of milk production. During this delicate postpartum period, mothers look to alternatives that can lead to breastfeeding cessation despite their preliminary goals and/or their family's goals.

Breastfeeding is the number one choice and recommendation according to the WHO (World Health Organization) and the CDC (Centers for Disease Control and Prevention). However, breastfeeding and chestfeeding parents may not meet the exclusive breastfeeding guidelines. Some reasons for earlier than desired cessation of breastfeeding include difficulties with lactation, birth outcomes like cesarean section or c-section, infant development including nutrition, weight, and reflexes, illness, medication side effects, lifestyle (e.g., returning to work), and the effort associated with pumping milk. Introducing the baby to formula (manufactured powered food) and/or utilizing a breast pumping device can alter the supply/demand relationship between lactating parent and baby.

Another known issue is that breast milk and formula milk can also contain undesirable or harmful contaminants that should not be passed onto the baby. Some of the various subtypes within the PFC family include (i) PFHxS (perfluorohexane sulfonate), (ii) PFOS (perflurooctane sulfonate), (iii) PFOA (perfluorooctanoic acid) and (iv) PFNA (perfluorononanoic acid). Each of the individual PFCs has unique properties in toxicokinetics and bio-elimination.

Unlike fat-soluble lipophilic compounds such as organochlorine pesticides, herbicides and polychlorinated biphenyls (PCBs), which accumulate in adipose tissue, perfluorinated compounds persist in bile in lean human tissue, including muscle, liver, and kidney. Much of the total body burden of PFCs remains in the blood bound to plasma proteins. According to the EPA, these chemicals build up internally over time. They can cause harm, based on laboratory animal testing, and/or include the low birth weight of newborn babies, child developmental disorders, and cancers. Animal and human studies have now linked PFC exposure with developmental toxicity, neurotoxicity, hepatotoxicity, carcinogenicity, metabolic dysregulation, immunotoxicity, and endocrine disruption.

Breast milk, while a carrier of nutritional components, may also be a carrier of toxic chemicals due to human and environmental exposure. Breast milk may contain chemicals belonging to a class of compounds known as PFAS at levels well above the safety thresholds set by governments, says the report from the international environmental group IPEN (International Pollutants Elimination Network). Long-term and short-term exposure to these chemicals can be linked but not limited to municipal waste dumps, food wrapping, home construction materials including carpets, textiles, kitchen pan non-stick coatings, fish, and even water.

Current studies show high levels of toxic PFAS chemicals in breast milk that far exceed U.S. drinking water health advisory limits. Additionally, human studies have also reported that PFCs (perfluorinated compounds) can cross the placenta and are present in breast milk). One study reported significant declines in serum PFOS in breastfeeding mothers, presumably due to vertical transmission of some PFCs into the infant via breast milk. As babies, specifically newborn babies, are their most fragile state, removing and limiting chemicals from breast milk is paramount.

Therefore, there is a need for the ability to selectively filter out harmful contaminants from both breast milk and formula milk, without filtering out any helpful nutrients or matter, before milk reaches the baby.

Breast milk can also contain microplastics and/or nano plastics, herbicides, and/or heavy metals which can cause inflammation, endocrine alterations, cell death, changes in the gut microbiome and cancer. The most common types of microplastics found in breast milk are polyethylene (PE), polyvinyl chloride (PVC), and polypropylene (PP). Toxic heavy metals like arsenic (a carcinogenic metalloid), mercury, lead and cadmium (chemical elements) can also be passed through breast milk. Formula milk can also contain micro and/or nano plastics, heavy metals due to compromised water tables, infrastructure (like lead piping), and pharmaceuticals (medications) in groundwater, surface water, and drinking water. Some pharmaceuticals or drugs that have been identified are antibiotics, antidepressants (SSRIs), antipsychotics, beta blockers, blood thinners, heart medications (ACE inhibitors, calcium-channel blockers, digoxin), hormones (estrogen, progesterone, testosterone), and painkillers, and more. Sewage treatment plants and facilities that treat water to make it drinkable are not currently designed to remove pharmaceuticals from water. While pharmaceuticals in the environment are problematic, drug entry into human milk vary by kinetic factors; the lipid solubility of the drug, the molecular size of the drug, the blood level attained in the maternal circulation, protein binding in the maternal circulation, oral bioavailability in the infant, and the mother, and the half-life in the maternal and infant's plasma compartments. Mothers/lactators may choose not to take medications or delay prescribed medications for fear of it passing through their milk to the baby. Bacteria and spores are also an issue in formula powdered milk. A 2022 powdered formula milk global supply chain crisis was compounded by a large scale infant formula recall in the United States of America after several babies allegedly died after consuming Abbott infant formula. This led to a Sturgis, Michigan manufacturing plant's production shutdown following the Food and Drug Administration recall of several more infant formula brands. The bacterium that sickened the infants, Cronobacter sakazakii, was found at the Sturgis plant.

Another known issue is “nipple confusion” or “nipple preference,” which describes an infant's fussiness at breast or frustration when they are having problems switching from a bottle nipple and breast, before breastfeeding is well established. A baby uses a completely different technique to remove milk from the breast than he uses to drink from a bottle. Some babies have difficulty alternating between a bottle and the breast and some do not. There is no way to predict who will have problems breastfeeding after drinking from a bottle. Babies that are born early or babies with a weaker or more uncoordinated suck may be more vulnerable to nipple confusion.

### BRIEF SUMMARY

The present disclosure is directed to a lactation flow meter device configured to accurately, in real-time, gauge or measure breast milk output. The device is directed at helping parents answer the question, “Am I making enough milk to sustain my baby?” The device is configured to promote lactation longevity, facilitate user benchmarks, provide early indicators of potential complications, empower breastfeeders and chestfeeders, and reduce anxiety and/or lessen stress through collected, processed, and displayed data.

The present disclosure is directed to a system or device and method for tracking, measuring, and providing breast milk production information or data. The collected or processed data includes flow rate, temperature, pressure, latch efficiency of the baby, and motion sensing of a baby's movements while breastfeeding, or any combination thereof. The device includes at least one sensor that is configured to collect the data.

The lactation flow meter device includes a flexible nipple shield adapted to conform to a shape of a teat. The shield includes a flow channel through which a volume of milk will operably pass from the nipple to a feeding baby. The device further includes a micro-fluidic flow meter to measure the milk output. The volume of milk passed from nipple to feeding baby is collected via an application (software stored in memory or a remote cloud-based database) for computer or mobile use (such as a tablet or a cell phone).

The battery housing can include a microprocessor or application specific integrated circuit (ASIC) that collects and processes the data gathered by the plurality of sensors, including timing information for collating the collected data and analyzing the data based on events that are detected simultaneously. The battery housing or the nipple shield can house a transmitter/receiver device that is configured to transmit wirelessly or in a wired manner the data collected by the plurality of sensors.

The data can be processed locally in the device or transmitted to a hand-held user device for processing and display to the user or mother. An application or software application is configured to receive all of the data and display information about a feeding session, like duration, flow rate for periods of time and as a whole, latch efficacy of the baby, or other suitable information derived from the data collected by the sensors.

In some embodiments, the device includes a filtration component configured to reduce any undesired or harmful chemicals, e.g., per- and polyfluoroalkyl substances (PFAS), perfluorooctane sulfonate (PFOS), perfluorooctanoic acid (PFOA), perfluorinated compounds (PFCS), herbicides, micro and nano-plastics, heavy metals, pharmaceuticals, and harmful bacteria like spores in breast milk and formula milk. The filtration component is a filter, cartridge, or sieve that can be worn on the breast, in the lactation flow meter device, within a vessel like a manufactured bottle, cup, container, or any combination thereof. The filter is removable to facilitate cleaning or replacement of the filter.

In some embodiments, a binder additive like molecular imprinted polygon (MIPs) is added to support the milk filtration. Binders create bonds between particles by chemical interactions like activated charcoal and zeolite.

In some embodiments, the device includes at least one latch sensor for optimal and suboptimal mouth latches benchmarked by the ‘L’ and ‘C’ in LATCH (LATCH is a breastfeeding charting system, see Terms and Definitions).

In some embodiments, the device is customizable and includes a personalized molded nipple shield, based on a custom mold of the human user's chest, adapted to operably conform in shape to the individual's breast or chest.

### BRIEF DESCRIPTION OF THE SEVERAL VIEWS OF THE DRAWINGS

FIG. 1A is a front view of a lactation flow meter device in accordance with an embodiment of the present disclosure.

FIG. 1B is a perspective side view of the device of FIG. 1A.

FIG. 1C is a perspective side view of the device of FIG. 1A with a removable filter.

FIG. 1D is a back view of the device of FIG. 1C.

FIG. 1E is a back perspective view of the device of FIG. 1C.

FIG. 1F is a back view of a nipple portion of the device of FIG. 1C.

FIG. 1G is a perspective view of the nipple portion of the device of FIG. 1F.

FIG. 2 is another embodiment of the lactation flow meter device.

FIG. 3 is an embodiment of a flow sensor.

FIG. 4 is another embodiment of the flow sensor.

FIGS. 5-7 are embodiments of a removable filter configured for use with a vessel.

FIG. 8 is an embodiment of the removable filter.

### DETAILED DESCRIPTION

Certain terms and definitions are set forth in order to provide a thorough understanding of the various embodiments of the present disclosure.

### Terms and Definitions

“Annubar,” “Pitot tube,” or “Pitot Probe” measures fluid flow velocity.
“Anemometer” measures the speed of wind or a gas stream (e.g., windmill or propeller).
“Binder or binding agent” is any material or substance that holds or draws other materials together to form a cohesive whole mechanically, chemically, by adhesion or cohesion.
“Breast” a soft protruding organ on the upper front of a body that secrete milk after prolactin and oxytocin are activated (e.g., birth, stimulation of nipples, or certain medications can induce).
“Breastfeeder,” “Breastfeeding,” “Lactator,” “Chestfeeder,” or “Chestfeeding” is a lactating individual that feeds from breasts or activity thereof. Chestfeeder is a lactating individual that may no longer have ‘breasts’ due to surgical reduction, hormone therapy, gender reassignment, or any combination thereof, but secretes milk from nipples.
“Breast milk” is milk produced by breasts after childbirth as food for offspring: a hormone called prolactin stimulates the body to produce breast milk, also known as human milk.
“Cartridge” is designed for insertion into a mechanism, device, or system.
“Cantilever low-flow liquid flow meter” is a cantilevered microstructure placed transversely within fluid flow. The cantilever deflection or inflection measures the flow rate.
“Glyphosate” is a non-selective herbicide (weedkiller marketed as Roundup®) and is detected in water and on land. Glyphosate has health concerns including cancer, harm to fetal growth and harm to kidneys.
“Human milk” see breast milk.
“Lactate” or “Lactating” means to secrete milk or the activity thereof.
“Lactator” is the person producing the milk secretions, traditionally but not always is the ‘birthing mother.’
“Lactation” is the process of making milk (the secretion of milk).
“Flowmeter” measures the flow rate of a liquid or gas; available in various configurations.
“Frenula” (singular) or “Frenulum” (plural) is of the mouth including the frenulum linguae under the tongue, the frenulum labii superioris inside the upper lip, the frenulum labii inferioris inside the lower lip, and the buccal frena, which connects the cheeks to the gum.
“Latch” is how well an infant latches on to the nipple/areola of a breast or chest to suckle, yielding milk extraction.
“LATCH” is a breastfeeding charting system that systematically gathers information about individual breastfeeding sessions. Each letter of the acronym LATCH denotes an area of assessment:
“L” is for how well the infant latches onto the breast.
“A” is for the amount of audible swallowing noted.
“T” is for the mother's nipple type.
“C” is for the mother's level of comfort.
“H” is for the amount of help the mother needs to hold her infant to the breast.
The system is visually represented in the same form as the Apgar (newborn test) scoring grid, and the numbers are handled similarly. With the LATCH system, a medical professional or nurse can assess maternal and infant variables, define areas of needed intervention, and determine priorities in providing patient care and teaching.
“Epidermal wearables” is a sector of the wearable industry experimenting with specific and targeted medical devices for healthcare applications; these skin-level implants focus on more specific applications. Due to their slim profile, they are ideal for extended wear, enabling long-term medical attention that could also be conducted remotely. Epidermal wearables are bandage-type wearable sensors that represent a rapidly emerging technology with considerable potential for low-cost decentralized (home or point-of-care) monitoring and diagnoses. Epidermal biosensors can detect biomarkers in bodily fluids like sweat, saliva, tears, and interstitial fluid (ISF). They can also analyze the skin surface itself.
“Filter” is a porous device for removing impurities or solid particles. The filter element includes a porous body for at least mechanically filtering breast milk and/or formula flowing into the filter element.
“Kinetic energy” is the energy possessed by a body by its motion.
“Microfluidic valve” also called “microvalve” are basic components used in microfluidic devices to handle liquids through different valve ports.
“Nipple” is the small projection in which the mammary ducts of female mammals terminate and from which milk can be secreted.
“Nipple Shield” is a thin material (often silicone) placed on the areola and nipple area.
“Osmosis” is the spontaneous passage or diffusion of water or other solvents through a semipermeable membrane.
“Paddle wheel flow meter” is a type of flowmeter using mechanical energy of the fluid to rotate a paddlewheel in the flow stream, creating rotational energy.
“PFAS,” “PFOS,” “PFOA,” or “PFCS” are a large, complex group of synthetic chemicals. These chemicals are considered man-made (not naturally occurring in the environment).
“Qi” is an interface standard for wireless power transfer using inductive charging.
“Radio Frequency” or “RF” can help enable the transfer and transmission of power across the wearer's skin's surface as an alternative to a traditional battery. This allows for continuous power transfer directly from a source on the body.
“Filter Screen” is a sieve with fine netting for filtration.
“Cable Sheath” is a protective covering around an electric cable.
“Teat” is a nipple of the mammary gland of a female mammal, from which the milk is sucked by the young or baby.
“Thermoelectric generators” (TEGs) are widely used for making wearables self-sustainable power generation. TEG devices can make electric power from waste heat (e.g., body heat).
“Toxin or detox binders” are specific compounds that bind to toxins and remove the toxins via the digestive tract so they are not reabsorbed into the body.
“Venturi flow meter” is a differential pressure flow meter that generates a flow measurement by measuring the pressure difference at two locations.
“Milk filtration” is the filtering of both breast/chest milk and formula.
“Molecularly-Imprinted Polymer (MIPs)” are synthetic polymers with a predetermined selectivity for a given analyte, targeting moiety, or group of structurally related compounds, making them ideal materials for separation processes. MIPs can be utilized to create sorbents that possess specialized binding capabilities to aid in filtration.
“Formula”—Infant liquid nutrition or supplement, given as a substitute for breast milk, or in tandem with breastfeeding called co-feeding.
“Gravity-feed” is making use of gravity to maintain the flow of material through a supply system.
“Reverse osmosis” pressure pumps force fluid through a membrane, which allows the pure fluid to pass through while filtering out contaminates like toxins, metals, plastics, bacteria and viruses.
A “Spore” is a reproductive cell produced by some plants, fungi and bacteria that can develop into a new individual. Some spores like Salmonella, Clostridium botulinum, and Cronobacter sakazakii can lead to infection and death in infants. Spore contamination has been found in powdered infant formulas.
“Weighted filter” is a flexible tube-like material with a weighted base that contains filter mechanism.
“Zeolite” is a family of several microporous, crystalline aluminosilicate materials commonly used as commercial adsorbents and catalysts.

### Lactation Flow Meter Device

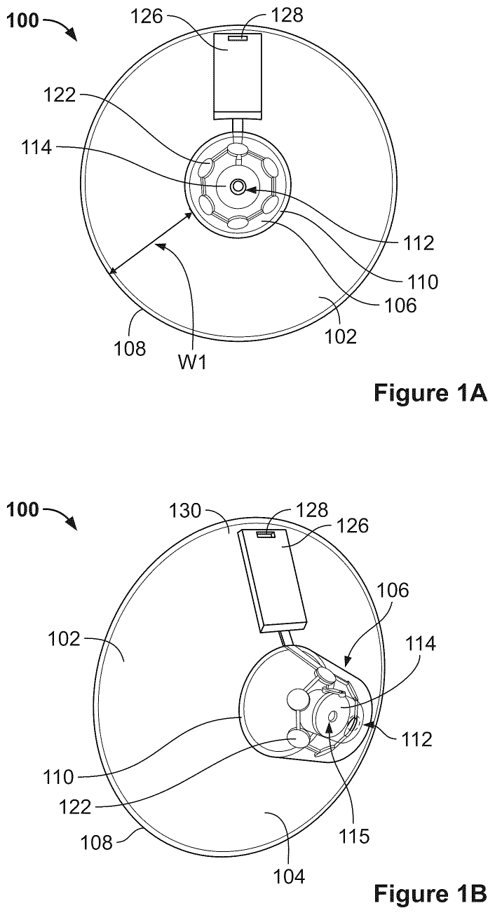

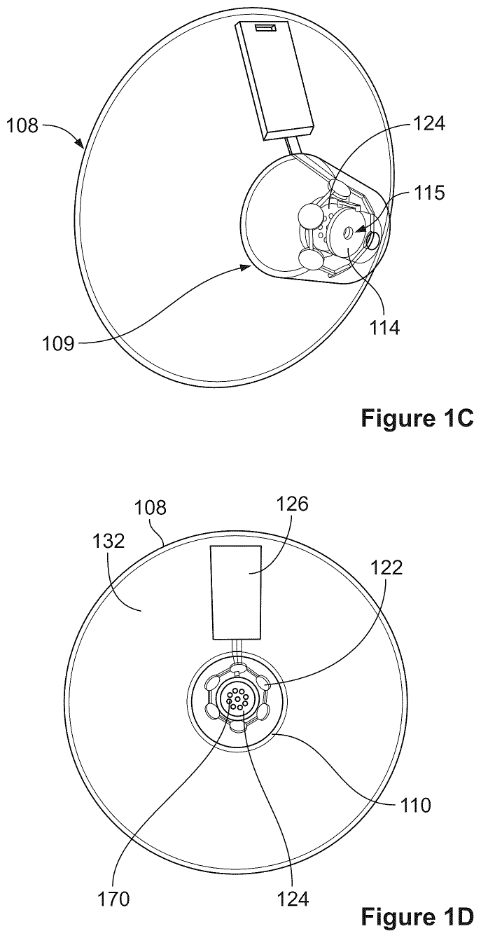

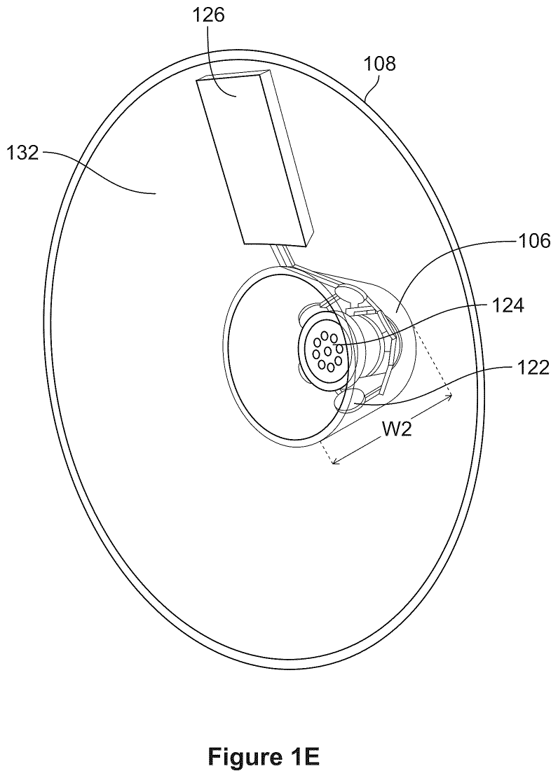

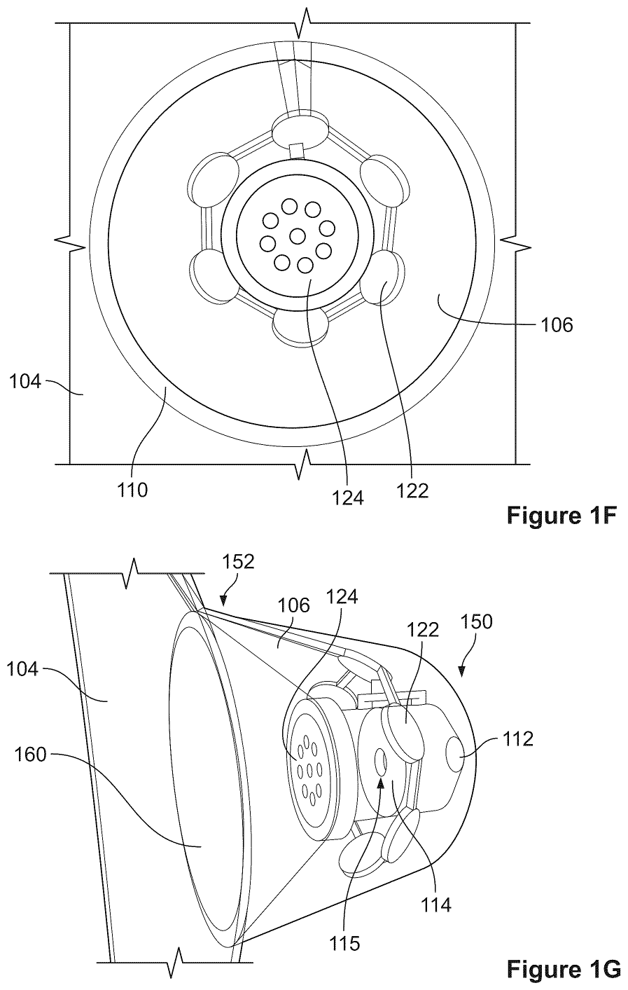

FIGS. 1A-1G are a lactation flow meter device or lactation device 100 that includes a nipple shield 102 having a chest or breast tissue support portion 104 and a nipple portion 106. The lactation flow meter device 100 is configured to detect and gauge a flow of breast milk moving through a channel of the nipple portion 106. The nipple portion 106 has a through opening 112 in fluid communication with an external environment. The nipple portion 106 includes at least one flow sensor 114 and at least one lactation sensor 122 configured to collect data. The sensors 114, 122 are electrically connected to a battery 126 in the support portion 104.

The device 100 is configured to detect the flow in real-time and provide the flow rate.

In some embodiments, the device 100 is a DTC (direct-to-consumer) device or a B2B (business-to-business) device.

### Nipple Shield

The nipple shield 102 has a front or exterior side 130 and an opposite back or interior side 132. The nipple portion 106 extending from the front side 130 that contacts the baby. The back side 132 contacts the user's chest.

The nipple shield 102 is made from rubber, silicone, also known as elastomer or polymer due to its non-reactive, flexible, and water-resistant properties, and is biocompatible, or other suitable oral-safe material for babies. The shape of the nipple shield 102 is customizable for each user to securely and comfortably support their breast tissue during use.

The nipple shield 102 includes the nipple portion 106 extending from a central region of the chest support portion 104. The support portion 104 has the outer edge or end 108 that is round and forms an outer diameter. A second inner end 109 is opposite the outer end 108. The second inner end 109 is round and forms an opening 160 having an inner diameter that is less than the outer diameter.

The support portion 104 has a curved portion or side extending from the outer end 108 to the inner end 109 for a dimension W1, as shown in FIG. 1A. The support portion 104 has a conical frustum-like shape or other suitable shape that comfortably conforms to and fits a user's breast or chest.

In this embodiment, the dimension W1 of the support portion 104 is greater than a dimension W2 of the nipple portion 106, as shown in FIG. 1E. In other embodiments, the dimensions W1, W2 are substantially similar or W1 is less than W2.

The nipple and chest support portions 106,104 are integral and contiguous.

The chest support portion 104 extends from an outer edge 108 to an interface 110 with the nipple portion 106. The nipple portion 106 has a first rounded end 150 opposite a second wider end or base 152. The nipple shield 102 includes at least one through opening 112 in the nipple portion 106. The opening 112 is spaced further from the outer edge 108 than the latch sensors 122 or battery 126. Said differently, the opening 112 is at a first end of the nipple shield 102 opposite the outer edge 108. A second through opening 160 is located at the base end 152 of the nipple portion 106. The second through opening 160 is wider and has larger diameter than the first opening 112 located at the rounded end 150.

In some embodiments, the back side 132 has a sticky or adhesive surface to attach to the user's skin.

In some embodiments, the support portion 104 has other suitable shapes and configurations.

In some embodiments, the opening 112 is centrally located in the nipple portion 106.

In other embodiments, the nipple portion 106 includes a plurality of openings 112. The plurality of openings 112 being spaced substantially equidistant from each other. In some embodiments, the plurality of openings 112 are in a circular or ring arrangement.

In some embodiments, the nipple portion 106 and the support portion 104 are manufactured independently or separately and then subsequently coupled together to form the nipple shield 102. In other embodiments, the nipple shield 102 is manufactured as a single piece comprising the nipple portion 106 and the support portion 104.

Although not shown, the enlarged view of the nipple portion 106 is similar to the device 100 of FIG. 1A.

### Flow Sensor

At least one flow sensor 114 is within the nipple portion 106. The flow sensor 114 is configured to measure or analyze milk flowing from the user through the nipple portion 106 to a feeding baby. The flow sensor 114 is a lactation flow meter configured to measure volume output, flow rate, or a combination thereof, of human milk.

FIG. 3 is an embodiment of the flow sensor 114 which is a microfluidic venturi meter with a first end 116 closer to the interface 110 than the opening 112 and a second end 118 adjacent to the opening 112.

The venturi meter measures the pressure drop across a smaller diameter opening, allowing flow measurement via the Bernoulli Equation. In this embodiment, the lactation device includes a plurality of through openings 112. A first opening of the plurality of through openings 112 having a different diameter than a second opening of the plurality of through openings 112. The first and second openings are opposite each other. The first opening is at a first end of the nipple portion 106 and the second opening is at an opposite second end of the nipple portion 106 located at the interface 110. The first and second openings are in fluidic communication with each other and an external environment.

The fluid or milk flows along a first direction from an end of the nipple portion 106 adjacent the interface 110 to another end of the nipple portion 106 adjacent the opening 112. The first end 116 of the flow sensor 114 has a first dimension along a second direction transverse the first direction. The second end 118 has a second dimension in the second direction. The flow sensor 114 includes a central portion 120 with a third dimension in the second direction. The third dimension is less than the first dimension and less than the second dimension. The central portion 120 is a constriction or restriction in a flow channel of the flow sensor 114. The second dimension, that is closer to the baby when in-use, may be less than the first dimension.

The nipple portion 106 of the device 100 in FIG. 1B extends from the chest support portion 104.

The flow sensor 114 includes a sensor positioned at the first end 116 and a sensor positioned at the second end 118 to detect a difference between the flow at the first end 116 and the second end 118. Each of these sensors may be a pressure sensor to detect pressure variations induced by the fluid passing by each sensor. The differential pressure measurement indicates the milk flow within the flow channel, as this differential can be inserted into the Bernoulli equation to solve for the volumetric flow rate.

In an alternative embodiment not shown, the flow sensor 114 is a paddle wheel or anemometer flow meter. The paddle wheel sits within the channel between the nipple placement and the end of the nipple shield 106. The secreted milk is expressed from the breast/chest through the force of the baby's suckling or sucking via a one-way paddle flow valve which induces sensor rotation. The rotations per minute (RPM) of the sensor correlates to the flow rate of the fluid. Increasing RPM indicates a higher fluid flow rate.

The paddle wheel flowmeter offers the ability to measure the flow of various compounds. Paddle wheel flowmeters are also known as the insertion flow meter or an inline flowmeter, the paddlewheel meter is propeller-shaped and compact. With computerized sensors, each paddle wheel meter is tested, and its accuracy is shown to be within one percent of a full-scale range reading. These parameters are printed and packaged with the purchased paddle wheel meter. The meter can be installed vertically or horizontally and should not affect performance.

In an alternative embodiment, a cantilever low-flow liquid flow meter or flow valve offers low-flow sensors through a fluid channel that detects ultra-low flow rates (e.g. nl/min). The symmetric position of the sensors flows in both directions and can be easily quantified. The increased velocity along the restriction induces a pressure drop measured with connected pressure sensors and then correlated with flow velocity and rate. These low-flow liquid flow meters are based on Bernoulli's laws from fluid mechanics, stating that flow velocity variations are correlated with pressure drops.

Cantilever low-flow liquid flow meters consist of microscale levers inserted perpendicularly into the fluid flow path. The micro-scale lever deflection measures the flow rate. As the flowing fluid moves the cantilever, piezoelectric microresistors determine the extent of movement and correlate this extent to a fluid velocity. FIG. 4 is an exemplary microcantilever-based gas flow sensor that is part of the flow sensor 114 of FIGS. 1A-1G. The flow sensor 114 includes a circular opening 115, 415 that is in fluid communication with the nipple portion 106 openings 112, 160 and external environment. The milk flows in the first direction, shown in FIG. 4 by an arrow, from a first side having the larger opening 160 through the flow sensor opening 115, 415 to a second side having the smaller opening 112 that faces the baby. The flow sensor includes at least one or a plurality of microcantilevers or cantilever beams 421 spaced from one another. Each microcantilever is a bent or curved projection extending in the first direction from an inner surface of a base or body of the flow sensor 417 to the smaller opening 112 of the nipple portion 106. Each cantilever 421 has a first end 423 opposite a second end 425; the first end of the cantilever 423 being substantially flat or straight and coupled to the flow sensor body 417; and the second end of the cantilever 425 being suspended in the flow sensor. The bent portion being adjacent the opening 415 of the flow sensor. The microcantilevers 421 being arranged around the flow sensor opening 415.

As milk moves from lactator to baby, the cantilever beams detect movement, indicative of flow or amount of milk moving to the baby; these signals are either processed in a chip in the shield device 100 and transmitted to the breastfeeder once the shield device 100 is coupled to an electronic device, like a phone or tablet; or can be wirelessly transmitted from an ASIC coupled to the sensor to the electronic device; the processing of the data can be performed to provide the mother with data about the flow rate of the milk. In other embodiments, other suitable mechanisms are used in the flow sensor 114 to sense or determine flow rate through the nipple portion 106.

In another embodiment, the first sensor is a pitot tube, which is a pressure differential flow sensor located in the flow channel of the nipple shield. Pitot tubes measure fluid velocity through a hollowed probe inserted parallel and counter currently into the moving fluid. This probe measures the pressure differential with respect to a reference pressure and determines the flowing fluid velocity via the Bernoulli Equation. The pressure measurement is relayed to the electronic circuitry to process and output the measurement data. The pitot tube may be inserted into the nipple shield 102 at a location such as 114 in FIG. 1. The electronic circuitry, the application-specific integrated circuitry (ASIC), receives signals from the sensor and outputs the data to a display device, e.g., a tablet, a laptop, a computer, a mobile phone, or another electronic device.

The pitot tube functions as an obstruction that changes the cross-section of the liquid flow in the pipe or conduit. In a differential pressure flow meter, as the liquid passes through the obstruction, its potential energy is converted into kinetic energy. The velocity of the liquid increases and is accompanied by a simultaneous decrease in the pressure. The velocity decreases when the liquid exits the obstruction, and the pressure increases again. This pressure drop generated across the obstruction is proportional to the square of the flow rate and is calculated using Bernoulli's equation.

An averaging pitot tube, also called an Annubar, is based on the traditional pitot tube design. While the pitot tube measures the pressure of a liquid at a single point, the more modern averaging pitot tube measures the average pressure by taking sample values at different points in the pipe. It is inserted directly into the pipe and perpendicular to the flow direction. The side of the tube facing the liquid flow consists of several impact pressure ports, while the opposite side may have single or multiple static pressure ports. These ports are connected together to a secondary device, such as a differential pressure transmitter.

When the liquid comes in contact with the tube, the kinetic energy of the liquid is converted into potential energy, and velocity is reduced to zero. The pressure now measured at the upstream ports is called the total impact pressure, which is the sum of the static pressure and the dynamic pressure of the liquid. The impact pressure is directly proportional to the flow rate of the liquid. As the liquid flows around the Pitot tube, the static pressure ports measure the decrease in pressure downstream of the flow.

### Battery

The device 100 includes a power source or battery 126 between the outer edge 108 and the interface 110. The battery 126 may be a lithium-ion battery or any other suitable energy source that can be easily replaced or charged like radio frequency (RF) that wirelessly transmits electricity. For charging, an electrical connection may be coupled to a receiving connection 128 accessible from an exterior surface 130 of the chest support portion 104.

In some embodiments, the power source 126 is a removeable, rechargeable, wireless, or any combination thereof.

The battery 126 may be embedded into the chest support portion 104, such as during a manufacturing process. In some embodiments, the chest support portion 104 is two separate sealed or coupled layers comprising an outer layer or surface 130 and an inner layer or surface 132. The two layers will be in direct contact in regions around the battery 126. The outermost layer 130 will include an opening that allows access to the receiving connection 128 for charging the battery 126. The battery 126 extends flat in or on the support portion 104 and extends between the outer edge 108 and the nipple portion 106.

In this embodiment, the battery 126 is positioned in an upper portion of the support portion 104, intended to be spaced from the baby. In other embodiments, the battery 126 is positioned anywhere in the support portion 104, such as a lower portion of the support portion 104.

The battery 126 is coupled to the flow sensor 114 by an electrical connection and provides power to electrical circuitry, computational equipment, potential micro-resistors, or any combination thereof.

The battery 126 is coupled to the plurality of latch sensors 122. Sensors 122 provide low-voltage signals that are interpreted by the computational circuitry. The collected or analyzed data is relayed to the user interface device. Battery remaining capacity data is also relayed to the user interface device.

Battery temperature is recorded via a Resistance Temperature Detector (RTD) or thermocouple and measurement is relayed to a processing computer.

The device 100 can be battery operated, which makes it highly portable, and can operate while the battery 126 is undergoing a charge cycle.

In an alternative embodiment, thermoelectric generators (TEGs) are used for charging power and sensors within the device 100. TEGs provide an energy source by directly converting low-grade heat to electricity. In some embodiments, flexible TEG systems are used, including thermoelectric (TE) films, thermoelectric bulks, printable thermoelectric inks, thermoelectric fibers, and organic thermoelectric materials.

In an alternative embodiment, the device 100 is powered through ion and electronic charge while in use or non-use. Additionally, Bluetooth connectivity and two-way Qi charging are the world's de facto wireless charging standard for providing 5-15 watts of power to small personal electronics and are compatible with all Qi chargers. Data is transferred via a Bluetooth chip or microchip module capable of broadcasting in the 2.4 GHz industrial, scientific, and medical (ISM) radio band.

### Latch Sensor

The device 100 includes at least one latch sensor 122. The lactation latch sensor 122 is configured to collect data about the strength, force, positioning, timing, and patterns of a baby's attachment to a nipple during feeding. Latch sensors 122 are configured to identify optimal and sub-optimal latches (e.g., deep latch vs. shallow latch). Latches can be possible indicators of anatomical issues in a baby's mouth.

In some embodiments, the device 100 includes a plurality of latch sensors 122. The latch sensors 122 are connected to each other. The latch sensors 122 have a disc or other suitable shape. The latch sensors 122 obtain data that can be processed and analyzed to provide personalized data to the user. The latch sensors 122 are arranged in a formation to provide means for obtaining lactation data. The latch sensors 122 are in the nipple portion 106 as seen in FIG. 1G. The latch sensors 122 have flat surfaces extending in along a direction of a surface of the nipple portion 106. the latch sensors 122 sense heat, pressure, motion, oral structure, or a combination thereof.

In one embodiment, there are six latch sensors.

In some embodiments, the plurality of latch sensors 122 are arranged substantially equidistant from each other in a ring formation around a cavity in the nipple portion 106 of the device 100.

Sensors 122 are devices that detect physical, chemical, and biological signals and provide a way for those signals to be measured and recorded. The physical properties of the sensors 122 are configured to utilize thermal properties, movements, and pressure to associate whether a baby has a shallow (nipple only) or deep (full areola) latch. Multiple latch pressure sensors 122 operate simultaneously to determine the depth and strength of the infant's latch. The sensors 122 emit signals that is read at the point of determination or transferred by wire or wireless transmission to remote locations (e.g., a personal mobile device, phone, tablet, computer and/or application).

In other embodiments, the device 100 does not include any latch sensors 122.

The device 100 collects data that is stored by the device 100, external computing device, software application, or any combination thereof. The associated weight gain and wet and dirty diapers will also be key indicators. The acquired data will mark the breast/chesting feeding relationship as successful. A suboptimal latch can be indicated by pain, issues with the breasts like inverted nipples, sore nipples, milk blebs (blisters), hotness, redness, inflammation, and infection like mastitis—all things the lactating parent may witness and experience. An approach to care for inverted nipples, sore and/or cracked nipples, and milk blebs is utilizing a nipple shell/shield.

Volume output, however, cannot be witnessed or experienced. If there is suboptimal volume output, then the lactation device 100 and latch sensor 122 will provide user benchmarks against ongoing datasets allowing for early indicators for ankyloglossia (tongue-tie) and/or lip-tie (tight oral frenula), cleft palate, micrognathia (recessed jaw), a weak suck, and other oral complications that can be brought on by anatomical mouth issues like Pierre Robin syndrome, or PRS, is a condition where babies are born with a small lower jaw, and/or genetic disorders like Cerebral palsy and Down syndrome due to poor muscle tone and weakness. This will prompt an alert within the device 100 or application and notify the user to seek medical help as determined by their provider and/or an opportunity to connect with a lactation professional, which may be coordinated through the device 100 or application. The device 100 or application receives and obtains lactation data that can be used to remotely monitor the user or patient. The lactation data can be analyzed or assessed by medical professionals, while maintaining standard medical compliance and regulations such as HIPAA, to remotely monitor their patients.

In some embodiments, the nipple shield 102 includes a microfluidic low-flow liquid meter gauge with optional latch sensors 122. In this embodiment, the device 100 includes an electronic interface and interconnect for measurement and display of milk flow and volume outputs during breastfeeding and optional removable filters configured to eliminate or reduce undesirable contaminants that are harmful to infants, such as PFAS/PFCs often referred to as “forever chemicals,” micro/nano plastics, heavy metals, pharmaceuticals and spores.

### Removable Filter

FIGS. 1C-1G are the device 100 including a removable filter 124, filtration cartridge, or other suitable filtration system, that is configured to filter out undesirable compounds, including, but not limited to, perfluorinated compounds and perfluoroalkyl substances.

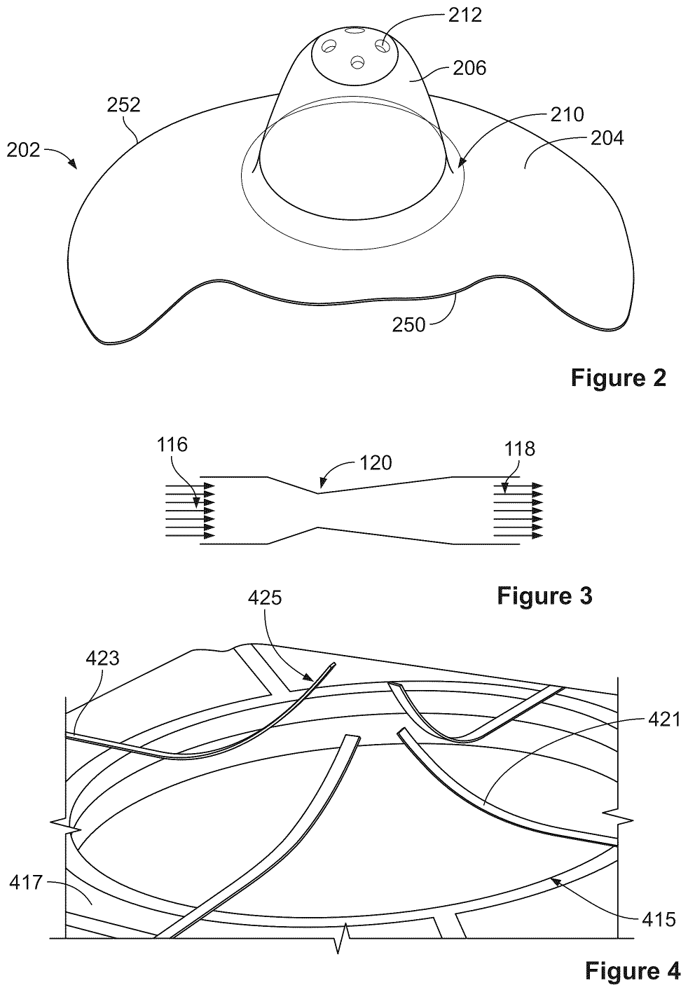

In some embodiments, the device 100 does not include the removable filter 124. FIGS. 1A, 1B, and 2 are devices 100, 202 according to this embodiment.

FIG. 1G is an enlarged view of the nipple portion 106 of FIG. 1C. The nipple portion 106 extends and tapers from the base 152 to the narrower first end 150. In other words, the nipple portion 106 has an inner funnel with a wider opening 160 at the base 152 tapering to a narrower opening 112 at the rounded end 150.

Filtration units that use granular activated carbon (GAC) or charcoal filters can effectively remove the PFAS compounds. In some embodiments, the nipple portion 106 includes a removable or replaceable filter that provides filtration for PFCs (perfluorinated compounds) and PFAs (perfluoroalkyl substances), and other harmful undesirable contaminants like micro/nanoplastics, herbicides, heavy metals, pharmaceuticals, and spores, to assist in removing toxins from breast milk.

In some embodiments, the removable filter 124 of the device 100 is removable from the back side 132 of the nipple shield 102. The back side 132 of the nipple shield 102 being the side that contacts the user or chestfeeder's skin or chest. In some embodiments, the removable filter 124 is a charcoal filter or other suitable material. The removable filter 124 is configured to fit within the nipple shield 102 of the device 100.

FIG. 1C is the removable filter 124 in the device 100. The removable filter 124 has a plurality of openings 170 in FIG. 1D. The plurality of openings 170 have a size and shape suitable for filtering the fluid or milk to remove undesirable particulate or contaminates. In some embodiments, the removable filter 124 has a disc or other suitable shape. The removable filter 124 in the nipple shield 102 is between the nipple portion 106 and the user's chest when the device is being used. The removable filter 124 is configured to purify the user's milk.

FIG. 1D is a back view of the lactation flow meter device of FIG. 1C. For illustration purposes, a back outer surface is not shown so the inner components can be more clearly seen. Near a center of the device is the removable filter 124. The removable 124 filter is surrounded by the plurality of latch sensors 122 that are in the nipple portion 106 of the device 100. The battery 126 is located in the support portion 106 of the device. The battery 126 has a first dimension from one end to another that extends adjacent the center or nipple portion 106 of the device 100 to the outer edge 108 or perimeter of the device.

Not shown, there is the back surface layer covering the back side of the device or support portion. The back surface layer covers the battery 126. The back surface layer contacts and protects the user's skin.

FIG. 1E is a perspective view of the device of FIG. 1D.

FIG. 1F is an enlarged back view of the nipple portion 106 of the device 100 of FIG. 1C.

FIG. 1G is a perspective enlarged view of the nipple portion 106 of the device 100 of FIG. 1C. An interior surface of the nipple portion 106 is sloped or conical. A first end of the interior surface of the nipple portion 106 is coupled to the support portion 104 of the device. The removeable filter 124 is at a second opposite end of the interior surface of the nipple portion 106. The interior surface tapers from the first end to the second end. The removable filter 124 is between the interior surface of the nipple portion 106 and a channel of the nipple portion. The channel has a through opening 160 that extends to the opening of the support portion 104. The opening 160 being central in a surface of the channel. Surrounding the channel are the plurality of latch sensors 122 that are coupled together and to the battery 126.

In an embodiment, lactation or feeding data is obtained, gathered, or determined while the device is offline via an “offline indirect” method. In this embodiment, the “offline direct” method obtains or derives a measurement without directly measuring flow or volume of fluid or milk. Rather, a calculated volume is presented or displayed only after the completed feeding session. In this “offline indirect” method, the pre-and post-measurement of the baby's weight is used to calculate the calculated volume. This method is currently endorsed by national health services.

In another embodiment, lactation or feeding data is obtained via a “Real-time indirect” method. In this embodiment, a proxy measurement is used to derive the delivered milk volume. The measurement may be relayed to the mother in real time. In some embodiments, this method includes measurement of the mother's breast milk volume via Doppler flow, skin-conductivity, physical volume, or other suitable measurement to determine lactation data. In one embodiment, the method does not involve breast measurement and instead relies on an acoustic signature of the baby's swallow in order to derive volume.

In another embodiment, lactation or feeding data is obtained via a “Real-time direct” method. The milk flow is measured directly as it passes from mother to baby, with the corresponding volume information being presented in real-time to the mother. A breast cup with a sensor mounted in or beside a milk channel which then leads to an artificial teat may be used. A variety of suitable sensor types may be used. In some embodiments, piezoresistive force, thermal gradient, mechanical turbine, reciprocating piston devices, or any suitable combination thereof are used.

In an embodiment, real time collection of data is obtained via a processor, processor unit or ASIC 140 located on or in the nipple shield 102. The processor 140 processes the lactation or feeding data.

In one embodiment, the processor 140 and battery source 126 is integrated or combined in a single unit within the nipple shield 102. The processor 140 receives raw data from the sensors 114, 122. In one embodiment, the raw sensor data is relayed to the processor 140 as a low voltage signal across microelectronic wires. Data is interpreted at the processor and relayed via Bluetooth to the mobile device. Data transferred via a Bluetooth chip or microchip module capable of broadcasting in the 2.4 GHz industrial, scientific, and medical (ISM) radio band.

In one embodiment, the nipple shield 102 has a customizable shape to fit the individual user. The custom shape may be obtained via 3D printing or other suitable methods to fit, mimic, or match the shape of the user's breast, chest, nipple, or teat. In some embodiments, 3D printing silicone may be used. The custom shape may be obtained via imaging or scanning of the user and using the obtained images or scans to create or manufacture the shape of the nipple shield 102. The custom shape may be generated using the obtained images or scans and configuring or generating the resulting custom shape using AutoCAD models.

In another embodiment, the custom shape of the nipple shield 102 is obtained using molds (molds of one's own breast/chest) to eliminate “nipple confusion”, to accommodate preferences, or a combination thereof. The custom shape of the nipple shield 102 allows toggling between shield and sans shield without causing confusion to the baby or otherwise interpreting or disrupting the feeding or lactation process.

A custom mold of one's breast/chest mimics the size and shape of nipple, areola, or combination thereof, helps to alleviate the confusion between switching from bottle, nipple shield and breast/chest. The mold of one's breast/chest are obtained by setting material application, 3D printing, use of AI (artificial intelligence), imaging, or other suitable means, or any combination thereof. Data about a plurality of breastfeeding sessions is saved in a memory, each with a time stamp and any other information provided by the user. Over time, with the compilation or addition of a plurality of data sets or obtained data, the data is processed to determine trends in the breastfeeding. In some embodiments, trends include an average over 7 days, days or times of day when the flow rate is above or below average, etc. In some embodiments, the data is stored on memory of the device. In other embodiments, the data is sent and stored on a personal device or other suitable memory storage device. The sensors will collect general biometric data backed against Lactation and Nutrition Guidelines. Daily benchmarks will be used to track and ensure the baby is getting proper intake. If suboptimal feeds are occurring, early intervention is key to avoid malnutrition and suboptimal latch, and possible pain and harm caused to the breast and nipple. Breast milk extraction (output) is recorded and stored to track daily minimum requirements. Helpful tips and access to lactation and clinicians via a software application on a portable device will also guide user experience and outcomes.

The primary benchmark to assess an optimal feed is volume output. If a baby is efficiently drawing milk from the breast and meeting the milk volume quota as quantified by the flow meters app (based on lactation and nutrition guidelines), then the user is notified of an optimal feed.

In some embodiments, the obtained data is used to further develop and improve the device, application, or both. Digital insights from the obtained data can be collected and analyzed to identify or assess trends, errors, issues, or other digital factors of the effectiveness, functionality, or accuracy of the device, application, or both. The digital insights can be used to identify areas of improvement for subsequent versions or iterations of the application or device. A microelectromechanical sensor can be included, such as a gyroscope or accelerometer to gather additional movement information about the breast feeding device.

In other embodiments, the device 100 collects the flow rate data during a breast-feeding session and then later wirelessly transmits the collected data to a display or portable electronic device, e.g., a tablet, a laptop, a computer, a mobile phone, or other suitable electronic device configured to receive, process, and store data.

FIG. 2 is another embodiment of a lactation flow meter device 202. In this embodiment, a nipple portion 206 has a plurality of openings 212. A portion of an outer edge 250 of a nipple shield follows a wave shape having protrusions and recesses. The outer edge 250 being on a first side or end of the device 202 opposite a second side or end 252. The wave shape extending for about half of the outer edge. Another portion of the outer edge being a substantially smooth curved edge. In some embodiments, there are four openings 212 spaced substantially equidistant from one another.

In other embodiments, the device 100 is an epidermal wearable or includes an epidermal wearable component. The epidermal wearable is configured to analyze the user, the feeding baby, including skin or other suitable biomarkers, breast milk, or any combination thereof. In some embodiments, a power source of the epidermal wearable uses radio frequency, for example, by transferring radio frequency energy from the user to the device.

### Filter Assembly Configured for Use with a Vessel

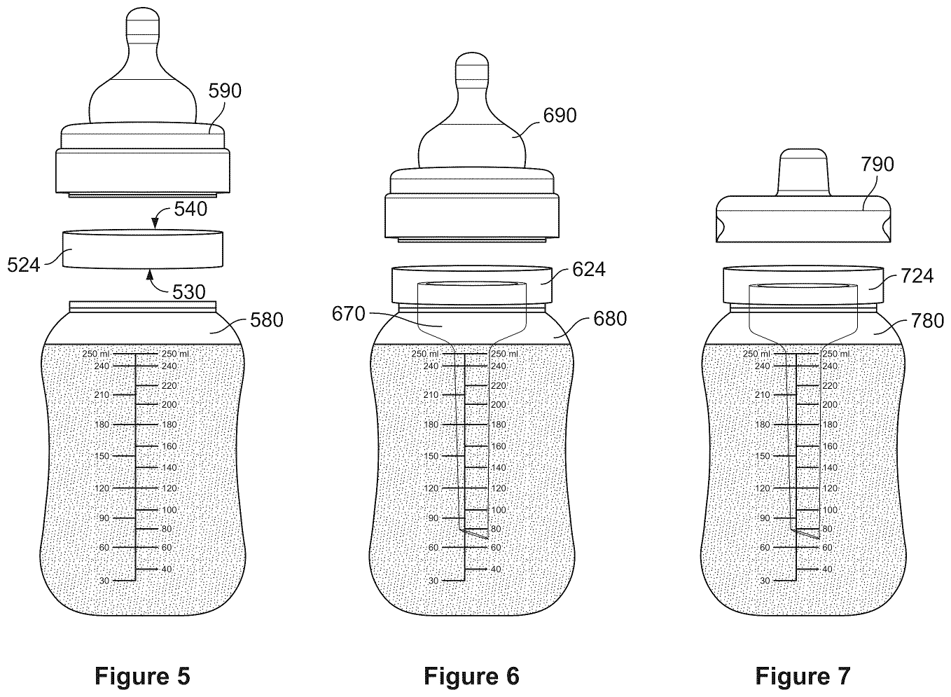

FIGS. 5-7 are embodiments of a filter assembly 524, 624, 724 configured for use with a vessel 580, 680, 780. The vessel is a bottle or container having an opening and configured to hold fluid such as breast milk or formula. The opening of the vessel extends along a first direction. The vessel has a removable lid or nipple 590, 690, 790 configured to be secured over the opening of the vessel. The nipple having ridges or other suitable mechanisms to create a seal when secured with the vessel.

FIG. 5 is an embodiment of the filter assembly 524 including at least one filter. The filter assembly 524 has a substantially disc shape, cylindrical shape, ring shape, or other suitable shape for securing the at least one filter.

The filter assembly 524 has a bottom or first side 530 configured to be secured to the vessel 580 in a way that fluid within the vessel 580 passes through the filter before exiting the vessel 580. The filter assembly 524 has a top or second side 540 opposite the bottom side 530. The top side 540 being configured to be secured to the nipple 590. The filter assembly 524 includes means for securing with the vessel 580, nipple 590, alone or in combination thereof. The filter assembly 524 means for securing are ridges or protrusions configured to fit and seal with securing means of the vessel 580 and nipple 590. In other words, the filter assembly 524 screws onto the vessel 580 and the nipple 590 screws onto the filter assembly 524. The filter assembly 524 is between the vessel and the nipple when in use and secured.

In other embodiments, the filter assembly is secured within the nipple or the vessel.

In some embodiments, the filter assembly 524 includes a housing or frame. In some embodiments, the housing is configured to secure the filter that is removeable. In other embodiments, the filter is coupled to the housing.

FIG. 6 is another embodiment of a filter assembly 624 having similar features as described in FIG. 5 and further includes a straw portion 670. The straw portion 670 is a tube, funnel, or other suitable elongated structure having a through opening or hole. The straw portion 670 has a first opening at a first end opposite a second opening at a second end.

The first opening has a larger diameter or area than the second opening. In other embodiments, the openings are substantially the same size and shape.

The straw portion 670 extends, along a second direction transverse the first direction, from a central region of the bottom side of the housing of the filter. The first end of the straw portion 670 tapers to the second end.

The filter assembly 624 is configured to secure with or within the vessel 680 so the straw portion 670 is entirely within the vessel 680. Fluid, including formula or breast milk, flows through the through opening to the lid 690 or external environment. The filter assembly 624 includes fastening or securing means configured to secure with the vessel 680, as described for FIG. 5.

In other embodiments, the housing portion of the filter assembly 624 is the first end of the straw portion 670.

In some embodiments, the filter assembly 624 further includes a pressure or compression mechanism to facilitate filtering. The compression mechanism is configured to evenly and consistently distribute pressure along the first direction across an internal area of the straw portion 670 or filter assembly 624. The internal area of the straw portion 670 is between sides of the straw portion 670.

In some embodiments, the compression mechanism includes a plunger, springs, or other suitable compression mechanism attached to the filter. The compression mechanism is in the straw portion 670.

In some embodiments, when the plunger is depressed, the filter seals or is adjacent to the second or bottom end of the straw portion 670. The filter has a central opening. The plunger coupled with the filter and extending through the central opening. Said differently, the filter is configured to move along the plunger within the straw portion 670.

In some embodiments, the compression mechanism is located within the housing of the filter assembly 624, the straw portion 670, or any combination thereof. In at least one embodiment, the compression mechanism begins in the housing of the filter assembly 624 and extends throughout the straw portion 670.

In other embodiments, the nipple and vessel have other configurations and shapes. FIG. 7 is another embodiment of a filter assembly 724 having similar features as described in FIGS. 5 and 6 and configured for use with a nipple having a shorter teat 790.

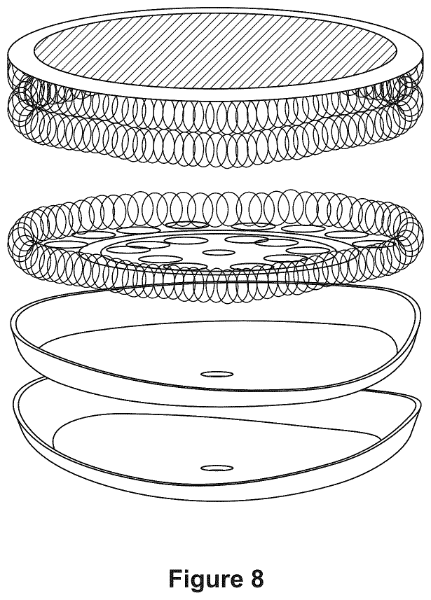

The filter assembly includes a filter having similar features and compositions as described above for FIGS. 1C-1G. The filter is made of a plurality of sub-layers. The sub-layers including at least two different materials or compositions, as shown in FIG. 8.

One of the sub-layers being a fine mesh layer made of metal or other suitable material. In one embodiment, the fine mesh layer comprises stainless steel. In other embodiments, this layer is absent.

Other sub-layers are made of charcoal, coconut husk, copper, other suitable filtering material that is food-safe and infant-safe, or any combination thereof. In other embodiments, this layer is absent.

Another sub-layer comprises cross-hatched fibers or interwoven material. In other embodiments, this layer is absent.

In other embodiments, the filter is a single layer of mixed material.

The filter is configured to filter chemicals, micro-and nano-plastics, harmful bacteria, and heavy metals from human milk (breast milk) and formula through a nipple shield, straw, drinking vessel, or other suitable device configured to hold, store, or pass-through milk.

In some embodiments, the filter is a stand-alone device. In other embodiments, the filter is configured for use with another vessel or device.

In some embodiments, the filter assembly is a removable or replaceable filter assembly. In other embodiments, the filter is a removable or replaceable filter.

Filtering is achieved through gravity-feed, charcoal, ceramic, carbon (like granular activated carbon), tablet, pouch, sieve, absorption, osmosis, ions, ultraviolet (UV), LED, manual pressure, suction, other suitable mechanisms, or any combination thereof.

In some embodiments, the filter is a weighted filter.

The various embodiments described above can be combined to provide further embodiments. Aspects of the embodiments can be modified, if necessary, to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A device, comprising:
a nipple shield having an outer edge, the nipple shield includes:
a first portion that includes an opening;
a second portion that extends from the first portion to the outer edge, the second portion being coupled to the first portion;
a first sensor positioned in the first portion, the first sensor including a first end adjacent to the opening, and a second end that is opposite to the first end; and
a plurality of latch sensors in the first portion and positioned between the opening and the second portion.

2. The device of claim 1 wherein the nipple shield includes a power source in the second portion, the power source is electrically coupled to the first sensor and the plurality of latch sensors.

3. The device of claim 1, wherein the first sensor is a flow sensor.

4. The device of claim 1, wherein the nipple shield has a customizable shape.

5. The device of claim 1, comprising a removable filter in the first portion.

6. A device, comprising:
a breast feeding shield that includes a breast coupling portion and a nipple portion, the breast feeding shield including:
an opening in the nipple portion;
a first flow rate sensor in the nipple portion; and
a plurality of latch sensors in the nipple portion.

7. The device of claim 6 wherein the opening is configured to allow milk to flow from the breast through the opening in response to a baby's sucking or nursing.

8. The device of claim 6 wherein the flow rate sensor includes a fluid path from the breast side to the nipple portion, the fluid path configured to allow milk to flow through, the flow rate sensor configured to detect a pressure or flow rate of milk from the mother.

9. The device of claim 6 wherein the nipple portion extends from the breast coupling portion and includes a recess extending in a first direction from the breast coupling portion.

10. The device of claim 9 wherein the fluid path is aligned with the first direction.

11. The device of claim 10 wherein the flow rate sensor includes a width in a second direction that is transverse to the first direction.

12. The device of claim 11 wherein the flow rate sensor includes a height along the first direction, the height being smaller than the width.

13. The device of claim 12 wherein the plurality of latch sensors includes a ring of sensors embedded in a material of the nipple portion.

14. The device of claim 13 wherein each one of the plurality of latch sensors includes a pressure sensor coupled to an adjacent one of the latch sensors with an electrical connection.

15. The device of claim 14 comprising a battery coupled to the latch sensor and the flow rate sensor.

16. The device of claim 15 wherein the battery is rechargeable.

17. The device of claim 16 comprising a filter in the recess and spaced from the opening by the flow rate sensor.

18. The device of claim 2, wherein the power source is a removable battery.

19. A device, comprising:
a removable filter assembly, including:
at least one filter;
a straw portion having a central through opening;
a plunger in the central through opening of the straw portion, the plunger is coupled to the at least one filter; and
a securing mechanism configured to secure with a vessel.

20. The device of claim 19, wherein the at least one filter includes a fine mesh layer.
